(19) United States
(12) Patent Application Publication
(10) Pub. No.: US 2020/0241004 A1
(43) Pub. Date: Jul. 30, 2020

(54) BIOMARKERS IN EX VIVO LUNG PERFUSION (EVLP) PERFUSATE

(71) Applicant: UNIVERSITY HEALTH NETWORK, Toronto (CA)

(72) Inventors: SHAFIQUE KESHAVJEE, TORONTO (CA); MINGYAO LIU, TORONTO (CA); MARCELO CYPEL, TORONTO (CA); ANDREW SAGE, TORONTO (CA)

(21) Appl. No.: 16/635,012

(22) PCT Filed: Jul. 30, 2018
(86) PCT No.: PCT/CA2018/050922

§ 371 Date: Jan. 29, 2020

Related U.S. Application Data
(60) Provisional application No. 62/654,738, filed on Apr. 9, 2018.
(60) Provisional application No. 62/539,373, filed on Jul. 31, 2017.

(51) Int. Cl. G01N 33/68

(52) U.S. Cl. CPC G01N 33/6893; G01N 33/6869; G01N 2333/5412; G01N 2333/70596; G01N 2333/70578; G01N 2800/245; G01N 2333/5421

(57) ABSTRACT

Methods and kits for screening, diagnosing, detecting or predicting a patient outcome/risk variable for a lung transplant recipient after transplant or an EVLP outcome by measuring biomarker levels of one or more biomarkers selected from IL-6, IL-8, sTNFR1 and sTREM-1 in EVLP perfusate are described. The methods involve for example, i. obtaining one or more test EVLP perfusate samples of a donor lung; ii. determining in one or more test EVLP perfusate sample of a donor lung, a polypeptide level of one or more biomarkers selected from IL-8, IL-6, sTNFR1 and sTREM-1; and iii. a) comparing the polypeptide level of the one or more biomarkers in the perfusate sample with a control or cut-off level, wherein the differential level is indicative of outcome/risk of after transplant or of an EVLP outcome; or b) using the polypeptide level of one or several of the one or more biomarkers in combination, as part of an algebraic calculation of outcome/risk.

### CROSS REFERENCE TO RELATED APPLICATIONS

This application claims the benefit of U.S. Provisional Application Ser. No. 62/539,373 filed Jul. 31, 2017, and 62/654,738, filed Apr. 9, 2018. All the teachings of the above-referenced applications are incorporated herein by reference.

### FIELD

The disclosure pertains to methods of assessing perfusate samples of donor lung grafts that have been submitted to Ex Vivo Lung Perfusion, for predicting patient outcome and for determining suitability for transplant.

### BACKGROUND

Ex vivo lung perfusion (EVLP) is a novel technique that was developed to prolong the normothermic assessment period of donor organs during lung transplantation (1-5). EVLP has been clinically validated and the technique is gaining widespread adoption worldwide. Currently, EVLP is hampered by a lack of predictive biomarkers that serve as reliable markers as to the process of EVLP, or the outcome of the organs that have been subject to EVLP during organ transplantation or after. Specifically, it is difficult to predict “patient outcome(s)” (PO) after transplant with lung having been subject to EVLP. Furthermore, many potential donor organs are placed on EVLP with the hope that they will improve and become suitable for transplant; however, in some cases the status of these lungs may not change and will ultimately be discarded following EVLP. The current selection of the healthiest donor lungs is based on a number of clinical findings including: donor type (brain and cardiac death donors), age, smoking history, ABGs, chest radiograph and bronchoscopic findings, as well as physical examination by the physician (6-8). Clinicians are very conservative during this examination and selection process, which results in a poor organ utilization rates.

The addition of a proteomic-based approach to assess donor organs during and after the EVLP procedure could allow for one or both of (i) prediction/determination of PO post-transplant and (ii) stratification of lung response to EVLP (for example to determine the likelihood of lung not improving with EVLP).

During the EVLP procedure, an acellular perfusate solution is circulated throughout the circuit. Upon the initiation of EVLP, there are no detectable levels of polypeptides specific to the lung in the acellular solution. As the procedure progresses, polypeptides are flushed and/or secreted from the donor organ and begin to accumulate in the perfusate. Identification and quantification of the various polypeptides and associated levels might provide a measure of health for the donor organ, which could be used to stratify potential donor lungs based on predicted response to EVLP and/or predicted PO.

Previous studies have shown that perfusate-derived polypeptide levels during EVLP could differentiate lungs that are transplanted with PGD Grade 0/1 compared to those that develop PGD Grade 3 after transplantation (9).

There is a need for the development of suitable biomarkers, kits, and compositions for screening for, diagnosing, detecting or predicting (i) EVLP outcomes and/or (ii) screening for, diagnosing, detecting or predicting PO after transplantation of EVLP treated lungs.

### SUMMARY

The inventors have identified several biomarker parameters that are differentially detected in EVLP treated donor lungs that are associated with one or more patient outcomes (PO) in transplanted patients and/or donor lung suitability for transplant after EVLP. Specifically, the inventors have identified key polypeptides that are present in EVLP perfusate that can be used to predict various aspects of PO including intensive care unit (ICU)-related length of stay and/or predict the lung's responsiveness to EVLP.

The disclosure provides in an aspect, methods for predicting patient outcome (PO) risk.

One aspect of the present disclosure is a method for the screening, diagnosing, or detecting of a outcome/risk comprising:
i. determining, in one or more test EVLP perfusate sample of a donor lung, the level of polypeptide of one or more biomarkers selected from the biomarkers of the invention; and
ii. a) comparing the level of the one or more biomarkers in the one or more perfusate sample with a control or cut-off level, wherein the differential biomarker level is indicative of outcome/risk of after transplant; or
   b) using the polypeptide level of one or several of the biomarkers in combination, as part of an algebraic calculation of outcome/risk of after transplant.

The outcome/risk can for example be the risk of a negative post-lung transplant patient outcome such as extended ICU length of stay, extended time on ventilator, extended post-transplant hospital stay.

In an embodiment, the method further comprises identifying a donor lung that has a decreased risk of having a negative post-lung transplant PO and optionally transplanting such donor lung into a suitable recipient.

In another embodiment, the method further comprises identifying a donor lung that has an increased risk of having a negative transplant outcome and optionally discarding the donor lung or using the donor lung for research or other purposes.

Another aspect of the present disclosure is a method for the early detection of a donor lung that will be declined at the end of the EVLP process comprising:
i. determining in a test EVLP perfusate sample of a donor lung the level of a polypeptide of one or more biomarkers selected from the biomarkers of the invention; and
ii. comparing the polypeptide level of the one or more biomarkers in the perfusate sample with a control or cut-off level, wherein the differential level is indicative of a lung that will be declined for transplantation; or
iii. using the polypeptide level in combination, as part of an algebraic calculation of outcome/risk.

In an embodiment, the method further comprises discarding the donor lung, and/or using the donor lung for research or other purposes if the one or more biomarker levels indicate that the lung will be declined for transplantation.

The disclosure also includes kits containing antibodies for the detection of the biomarkers of the invention that are used to measure the biomarker polypeptide levels.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments of the disclosure are described with reference to the drawings:

FIG. 1 shows a graph depicting relationship between patient intensive care unit (ICU) stay and PGD phenotype. Box and whisker plots for patient PGD status (the solid line represents the median and the dashed line represents the mean ICU stay). Cases were either PGD 0/1 (NON-PGD), PGD2, PGD3, or determined to be clinically ‘Excluded’ from PGD grading. Each circle represents a single transplant case and the dashed line represents a two-week (14-day) ICU stay.

FIG. 2 shows a graph depicting relationship between patient ICU stay and extubation time. Correlation between a transplant patient's ICU length of stay and days to extubation. Each point represents a single transplant case, symbols represent PGD status, and the dashed line represents a two-week (14-day) ICU stay. r, r², and p values are shown above the plot.

FIG. 3 shows graphs depicting relationship between total patient hospital stay and (A) ICU length of stay or (B) post-transplant ICU length of stay. Each circle represents a single transplant case and the correlation parameters (r, r², and p values) are shown above the plot.

FIG. 4. shows graphs depicting relationship between patient ICU length of stay and post-transplant ICU length of stay (A) or mechanical ventilator days (B). Each circle represents a single transplant case and the correlation parameters (r, r², and p values) are shown above the plot.

FIG. 5 shows graphs depicting relationship between ICU length of stay and the rate of CXCL8 (A) or IL-6 (B) production during EVLP in DCD donors. Each circle represents a single transplant case; correlation coefficient and p-values are indicated on each graph.

FIG. 6 shows a graph depicting univariate prediction of transplant related ICU stay greater than two weeks (14 days) against cases with an ICU stay less than two weeks (14 days) against the null base model. Box and whisker plots for the AUCs for each EVLP biomarker with 100×10-fold cross validation shown as open circles and the test on training set AUC is shown as a diamond.

FIG. 7 shows a graph depicting univariate prediction of transplant related ICU stay greater than two weeks (14 days) with pre-transplant ICU stay cases removed against the base clinical model of donor type. Box and whisker plots for the AUCs for each EVLP biomarker with 100×10-fold cross validation shown as open circles and the test on training set AUC is shown as a diamond.

FIG. 8 shows a graph depicting multivariate predictions of EVLP cases with a transplant-related ICU stay greater than two weeks (14 days). Box and whisker plots for the AUCs for each EVLP biomarker combination are shown on the x-axis as open circles with the dashed line representing the AUC of the best univariate model.

FIG. 9 shows a graph depicting univariate prediction of EVLP cases declined for transplant against cases with an ICU stay less than two weeks (14 days) against the null base model. Box and whisker plots for the AUCs for each EVLP biomarker with 100×10-fold cross validation shown as open circles and the test on training set AUC is shown as a diamond.

FIG. 10 shows a graph depicting univariate prediction of EVLP cases declined for transplant against cases with an ICU stay less than two weeks (14 days) against the clinical base model (donor type and donor gender). Box and whisker plots for the AUCs for each EVLP biomarker with 100×10-fold cross validation shown as open circles and the test on training set AUC is shown as a diamond.

FIG. 11 shows a graph depicting multivariate prediction of EVLP cases declined for transplant against cases with an ICU stay less than two weeks (14 days). Box and whisker plots for the AUCs for each EVLP biomarker combination are shown on the x-axis as open circles with the dashed line representing the AUC of the best univariate model.

FIG. 12 shows a graph depicting multivariate prediction of EVLP cases declined for transplant against cases with an ICU stay less than two weeks (14 days). Shown are Box and Whisker plots of 100×10-fold cross validation of AUCs for each EVLP biomarker combination. Each combination shown was better than the base model (clinical model (donor type, donor gender, donor age) with the best univariate predictor (CXCL8 at 4HR)) shown on the x-axis.

FIG. 13 shows a graph depicting relationship between sTNFR1 and PO. sTNFR1 levels (pg/mL) corrected for lung size (total lung capacity (TLC)) after 1 hour of EVLP and separated based on PO (short ICU stay (white bar) and long ICU stay (black bar)).

FIG. 14 shows univariate analysis of IL-6 levels at 4 hours (normalized to TLC). Shown are the areas under the ROC curve (AUROC) for good PO compared with bad PO for several donor characteristics including: gender, type (DBD or DCD), body mass index (BMI), age, smoking history.

FIG. 15 shows graphs depicting interval sampling of IL-6 during EVLP. (A) IL-6 levels during EVLP measured every 15 minutes for a declined (x) and transplanted (o) case. (B) Regression analysis of IL-6 levels measured every 15 minutes during EVLP and extrapolated to 4 hours (240 minutes). r² values are shown for a transplanted (o) and declined (x) case.

FIG. 16 shows use of single lungs to predict double lung outcomes. Shown are the measured IL-6 levels after 4 hours EVLP for a right lung only (with good PO) and left lung only (with good PO). A theoretical double lung (right and left) can be calculated (44 ng/mL) and compared to the actual, measured, IL-6 values of double lungs with good PO (40 ng/mL).

### DETAILED DESCRIPTION OF THE DISCLOSURE

### I. Definitions

The term “patient outcome” also referred to as “outcome” as used herein means one or more of primary graft dysfunction (PGD) grade, graft-related patient death, total hospital length of stay, transplant-related hospital length of stay, total intensive care unit (ICU) length of stay, transplant-related ICU length of stay, post-transplant ICU length of stay, days on mechanical ventilation, patient-related use of extracorporeal membrane oxygenation (ECMO).

The term “biomarkers of the invention” as used herein means one or more of sTREM-1 (soluble (TREM1)), IL-6 (IL6), IL-8 (CXCL8) and sTNFR1 (soluble (TNFRSF1A)).

The term “polypeptide” as used herein refers to a polymer consisting a number of amino acid residues bonded together in a chain. The polypeptide can form a part or the whole of a protein. The polypeptide may be arranged in a long, continuous and unbranched peptide chain. The polypeptide may also be arranged in a biologically functional way. The polypeptide may be folded into a specific three dimensional structure that confers it a defined activity. The term “polypeptide” as used herein is used interchangeably with the term “protein”.

The term “soluble TREM1” or sTREM-1 as used herein means non-cell bound forms of Triggering receptor expressed on myeloid cells and includes all naturally occurring cleaved or released forms, for example from all species and particularly human including for example human sTREM-1 which has at least the extracellular portion of sTREM-1, for example amino acid 21 to 205 of accession number Q9NP99, herein incorporated by reference.

The term “IL-6” or “IL6” as used herein means interleukin-6 which is a secreted cytokine, and includes all naturally occurring forms, for example from all species and particularly human including for example human IL-6 which has amino acid sequence accession P05231, herein incorporated by reference.

The term “IL-8” also referred to as CXCL8, as used herein means interleukin-8 which is a secreted cytokine, and includes all naturally occurring forms, for example from all species and particularly human including for example human IL-8 which has amino acid sequence accession P10145, herein incorporated by reference.

The term “sTNFR1” or “soluble (TNFRSF1A)” used herein means non-cell bound forms of tumor necrosis factor (TNF) receptor superfamily member 1A, and includes all naturally occurring cleaved or released forms, for example from all species and particularly human including for example human sTNFR1 which has at least the extracellular portion of TNFR1, for example amino acid 22 to 211 of accession number P19438, herein incorporated by reference.

The term “donation after cardiac death” or “DCD” as used herein means the withdrawal of life support of a patient after it has been determined that there is no long-term prognosis for recovery, and subjects who experience cardiocirculatory arrest and a qualified decision is made to terminate or not initiate resuscitation. A DCD lung graft is accordingly a lung graft obtained from such a patient. DCD is also meant to include NPOD (non-perfused organ donor) and uDCD (uncontrolled (Maastricht) DCD) donors.

The term “donation after brain death” or “DBD” as used herein means donors who experience the irreversible end of all brain activity but whose body, including transplantable organs, are maintained through external mechanical means.

The terms “control” and “cut-off level” as used herein respectively refer to a control graft with known outcome and a predetermined threshold value based on a plurality of known outcome grafts, and for biomarkers associated with increased polypeptide level in poor grafts e.g. those with poor PO or which are likely to be declined following EVLP, above which threshold a graft is identified as having an increased risk of developing poor PO post-transplant and/or being declined after EVLP and below which (and/or comparable to) a candidate donor lung is identified as having a decreased risk of developing poor PO post-transplant or being declined post EVLP. The threshold value can for example for each of the one or more polypeptide biomarkers of the invention, be determined from the levels or parameter values related thereto of the biomarkers in a plurality of known outcome lungs. For example, an optimal or an acceptable threshold can be selected based on the desired tolerable level of risk. The cut-off level may for example depend on the PO being assessed. For example, for the patient outcome “total ICU stay”, patients that have pre-transplant ICU stay are typically sicker and would also have longer post-transplant ICU stays. The cut-off level may also for example include donor characteristics, for example gender, type (DBD or DCD), age, body mass index (BMI), and/or smoking history. Accordingly, the biomarker ‘cut-off’ can in some embodiments be adjusted for patients who have different duration of ICU stays, and donor characteristics.

The term “good outcome lung grafts” as used herein means lung grafts that are predicted to be and/or which are characterized as being suitable for clinical transplantation after EVLP and/or which result in a good PO in the recipient after transplantation. For example, good PO could include being free from: graft-related death causes within 30 days, PGD3, extracorporeal life support/ECMO, prolonged hospital/ICU stays or prolonged time spent on a mechanical ventilator.

The term “poor outcome lung grafts” as used herein means lung grafts that are predicted to be and/or which are characterized as being less or unsuitable for clinical transplantation after EVLP or, in the recipient after transplantation, inducing poor PO such as death from graft-related causes within 30 days, PGD3, requiring extracorporeal life support/ECMO, prolonged hospital/ICU stays, or time on mechanical ventilation. Examples of a poor-PO graft include a graft that after transplanting would result in a patient requiring an ICU stay greater than two-weeks (14 days), as well as a graft that has an increased risk of having a PGD3 lung transplant outcome. A lung graft can be characterized as being unsuitable for clinical transplant after EVLP for example after visual and physiologic examination such as when gas exchange function is not acceptable represented by a partial pressure of oxygen less than 350 mmHg with a fraction of inspired oxygen of 100%; or 15% worsening of lung compliance compared to 1 h EVLP; or 15% worsening of pulmonary vascular resistance compared to 1 h EVLP; or worsening of ex vivo x-ray. The assessment of suitability for transplant requires significant skill and experience. Biomarkers that are able to predict suitability can provide a more accessible quantitative benchmark for use in assessing transplant suitability.

The term “Acute Physiology And Chronic Health Evaluation Score” or “APACHE score” as used herein refers to an initial risk classification system for severely ill hospitalized patients. For example, it is applied within 24 hours of admission of a patient to an ICU. An integer score is computed based on several measurements, and higher scores correspond to more severe disease and a higher risk of death. For example, the point score is calculated from a patient's age and 12 routine physiological measurements: AaDO₂ or PaO₂ (depending on FiO2); temperature (rectal); mean arterial pressure; pH arterial; heart rate; respiratory rate; sodium (serum); potassium (serum); creatinine hematocrit; white blood cell count; and Glasgow Coma Scale. The score can also take into account of whether the patient has acute renal failure, and whether prior to hospital admission the patient has severe organ system insufficiency or is immunocompromised.

The term “perfusate sample” as used herein means an aliquot of a perfusion solution such as Steen Solution™ that is used for EVLP and which is taken subsequent to starting EVLP, for example after at least or at about 45, 60, 75, 90 and/or 105 min, and/or after at least or at about 2, 2.5, 3, 3.5, 4, 4.5, 5, 5.5 and/or 6 hours subsequent to starting EVLP, or optionally at time of fluid replenishment or any time between 45 min and 6 hours, optionally between 1 hour and 4 hours or any increment of 1 minute, 5 minutes or 15 minutes or any time therebetween. The term “perfusate sample” and “EVLP perfusate sample” are used interchangeably in the present disclosure. Perfusate samples can be used directly or snap frozen for later testing. The perfusate sample can, for example, be purified and/or treated prior to assessment.

The term “perfusion solution” as used herein means a buffered nutrient solution that can be used for EVLP, including for example STEEN Solution™. STEEN Solution™ is a buffered extracellular solution developed specially for EVLP that contains Dextran 40, human serum albumin and extracellular electrolyte composition (low K+) that provides cellular/organ protection and optimized colloid osmotic pressure. The skilled person can readily recognize that the perfusion solution can be any buffered nutrient solution that is suitable for and/or supports ex vivo lung perfusion for lungs that may be used for transplantation.

The term “declined lungs” as used herein means lungs that after EVLP are declined for transplant. Such lungs can discarded and/or used for research or other purposes. Lungs are presently typically declined for example if gas exchange function is not acceptable, represented by a partial pressure of oxygen less than 350 mmHg with a fraction of inspired oxygen of 100%; or 15% worsening of lung compliance compared to 1 h EVLP; or 15% worsening of pulmonary vascular resistance compared to 1 h EVLP; or development of significant edema; or worsening of ex vivo x-ray. As described herein, lungs are declined during or at the end of the EVLP process if comparison between parameter values related to polypeptide levels of biomarkers from EVLP perfusate samples of a donor lung with a control or cut-off level determines that the donor lung is to be declined for transplantation. The biomarker can be any biomarker described herein, including a polypeptide selected from IL-8, IL-6, sTNFR1 and sTREM-1, preferably comprising at least one of IL-8 and IL-6 in the test EVLP perfusate samples. The assessment can involve the generation of transplant suitability score for the donor lung based on the parameter values derived from the polypeptide level of the one or more biomarkers.

The term “suitability for transplant” as used herein means an organ that is predicted to be a good PO lung graft, for example to have a decreased risk of a prolonged ICU stay post-transplant.

The term “PGD3” as used herein means Primary Graft Dysfunction Grade 3 as defined by the standardized consensus criteria of International Society for Heart and Lung Transplantation (ISHLT) or similar.

The term “antibody” as used herein is intended to include monoclonal antibodies including chimeric and humanized monoclonal antibodies, polyclonal antibodies, humanized antibodies, human antibodies, and chimeric antibodies. The antibody may be from recombinant sources and/or produced in transgenic animals. The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, diabodies, bispecific antibody fragments and other fragments can also be synthesized by recombinant techniques. The skilled person can readily recognize that a suitable antibody for the invention is any antibody useful for detecting biomarkers described herein in any detection method described herein. For example, useful antibodies include antibodies that specifically bind to IL-6, IL-8, sTREM-1 or sTNFR1 polypeptide.

The term “detection agent” refers to an agent (optionally a detection antibody) that selectively binds and is capable of binding its cognate biomarker compared to another molecule and which can be used to detect a level and/or the presence of the biomarker. A biomarker specific detection agent can include probes and the like as well as binding polypeptides such as antibodies which can for example be used with immunohistochemistry (IHC), Luminex® based assays, ELISA, immunofluorescence, radioimmunoassay, dot blotting, FACS, protein microarray, Western blots, immunoprecipitation followed by SDS-PAGE immunocytochemistry Simple Plex assay or Mass Spectrometry to detect the polypeptide level of a biomarker described herein. Similarly, “an antibody or fragment thereof” (e.g. binding fragment), that specifically binds a biomarker refers to an antibody or fragment that selectively binds its cognate biomarker compared to another molecule. “Selective” is used contextually, to characterize the binding properties of an antibody. An antibody that binds specifically or selectively to a given biomarker or epitope thereof will bind to that biomarker and/or epitope either with greater avidity or with more specificity, relative to other, different molecules. For example, the antibody can bind 3-5, 5-7, 7-10, 10-15, 5-15, or 5-30 fold more efficiently to its cognate biomarker compared to another molecule. The “detection agent” can for example be coupled to or labeled with a detectable marker. The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

The term “level” as used herein refers to an amount (e.g. relative amount or concentration as well as parameter values calculable based thereon such as a rate or ratio) of biomarker (i.e. polypeptide related level) that is detectable, measurable or quantifiable in a test biological sample and/or a reference biological sample, for example, a test perfusate sample and/or a reference perfusate sample. For example, the level can be a rate such as pg/mL/hour, a concentration such as μg/L, ng/mL or pg/mL, a relative amount or ratio such as 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 10, 15, 20, 25, and/or 30 times more or less than a control biomarker or reference profile level. The control biomarker polypeptide level can, for example, be the average or median level in a plurality of known outcome lungs. Parameter values related to a level include concentration, rate of production and a ratio or fold increase (e.g. concentration at a later time point (such as 4 hours) divided by a concentration at an earlier time point for the same biomarker).

The term “subject” as used herein includes all members of the animal kingdom including mammals, and suitably refers to humans.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 50%, 5-50%, or 10-40%, 10-20%, 10%-15%, preferably 5-10%, most preferably about 5% of the number to which reference is being made.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### II. Methods

Disclosed herein are polypeptide biomarkers that can be used to assess whether a lung graft such as a marginal lung graft that is subjected to normothermic ex vivo lung perfusion (EVLP) is suitable for transplant.

The inventors have identified several biomarkers that are differentially expressed in donor lungs that are associated with, and can be used to predict patient outcomes (PO) post-transplant. Specifically, the inventors have identified key polypeptides that are present in EVLP perfusate that predict intensive care unit (ICU) length of stay and/or a declined donor organ. The disclosure provides in an aspect, methods for diagnosing patient risk and identifying declined lungs early in the EVLP process.

One aspect of the present disclosure is a method for the screening, diagnosing, or detecting the outcome/risk as it relates to donor lungs such as a risk of a prolonged ICU stay comprising:
i. determining in one or more test EVLP perfusate samples of a donor lung the level of one or more biomarkers selected from the biomarkers of the invention; and
ii. comparing the level of the one or more biomarkers in the perfusate sample with a control or cut-off level, wherein the differential biomarker level is indicative of the outcome/risk after transplant; or.
iii. using the polypeptide level of several polypeptides in combination, as part of an algebraic calculation of outcome/risk after transplant.

Also provided in another aspect a method for predicting a patient outcome variable for a lung transplant recipient after transplant, the method comprising:
i. obtaining one or more test EVLP perfusate samples of a perfusion solution collected during perfusion of a donor lung;
ii. measuring in the one or more test EVLP perfusate samples one or more parameter values related to a level of one or more biomarkers selected from IL-8, IL-6, sTNFR1 and sTREM-1;
iii. optionally generating a PO variable score for the donor lung based on the one or more parameter values; and
iv. comparing one or more parameter values or optionally the PO score for the donor lung with a control or cut-off level, wherein the PO variable score is indicative of a PO variable after transplant.

In an embodiment, the outcome/risk (also referred to as patient outcome related risk) or PO variable is selected from ICU length of stay, post-transplant hospital length of stay, number of days on a ventilator, APACHE score and post graft dysfunction (PGD) grade, optionally PGD0/1 or PGD3.

In an embodiment, the outcome/risk is determined to be acceptable or sufficient to discontinue EVLP.

In an embodiment, the outcome/risk or PO variable is total ICU stay.

In an embodiment, the outcome/risk or PO variable is ICU post-transplant length of stay.

In another embodiment, the outcome/risk or PO variable is post-transplant hospital length of stay.

In yet another embodiment, the outcome/risk or PO variable is number of days on a ventilator.

In another embodiment, the outcome/risk or PO variable is APACHE score.

In another embodiment, the outcome/risk or PO variable is post graft dysfunction (PGD), optionally PGD0/1 or PGD3.

In an embodiment, the method further comprises identifying or selecting donor lungs that have a decreased risk of having poor PO post lung transplant. In an embodiment, the donor lung is selected, prepared for and optionally transplanted into a suitable recipient if the outcome/risk or PO variable is acceptable. For example the donor lung is suitable, if the level, combination of levels, parameter values, PO-score or transplant suitability score, are below the selected cut-off value.

The algebraic calculation, PO-score or transplant suitability score can be calculated using a computer.

In another embodiment, the method further comprises identifying a donor lung that has an increased risk of having a negative transplant outcomes and optionally discarding the donor lung or using the donor lungs for research or other purposes if the outcome/risk or PO variable is unacceptable or above the cut-off value. For example the donor lung is declined, if the level, combination of levels, parameter values or score are above the cut-off value.

A further aspect relates to early detection of donor lungs that will be ultimately declined after EVLP. Clinical EVLP is typically approximately 4 to 6 hours. As demonstrated herein, the inventors have identified biomarker parameter values that can identify donor lungs that would likely be declined after EVLP earlier in the EVLP process, for example after at least or at about the 1 hour mark. The earlier identification is a benefit for example in time and resources.

Accordingly another aspect of the present disclosure is a method for the early detection of donor lungs that will be declined at the end of the EVLP process comprising:
i. obtaining one or more test EVLP perfusate samples of a perfusion EVLP solution collected during perfusion of the donor lung;
ii. determining in the one or more test EVLP perfusate samples the level of one or more biomarkers selected from the biomarkers of the invention; and
iii. comparing the level of the one or more biomarkers in the perfusate sample with a control or cut-off, wherein the differential biomarker level is indicative of a lung that will be declined for transplantation; or
iv. using the polypeptide level of several polypeptides in combination, as part of an algebraic calculation to determine lungs that will be declined for transplantation.

In an embodiment, the method comprises:
i. obtaining one or more test EVLP perfusate samples of a perfusion EVLP solution collected during perfusion of the donor lung;
ii. determining one or more parameter values related to a level of one or more biomarkers selected from IL-8, IL-6, sTNFR1 and sTREM-1 in the one or more test EVLP perfusate samples;
iii. optionally generating a transplant suitability score for the donor lung based on the one or more parameter values;
iv. comparing the one or more parameter values or optionally the transplant suitability score for the donor lung with a control or cut-off level, and
v. continuing perfusion if the one or more parameter values or transplant suitability score indicates that the donor lung is suitable for transplantation and discontinuing perfusion if the one or more parameter values or the transplant suitability score indicates that the donor lung will be declined for transplantation.

In another embodiment, the method further comprises discarding the donor lung and/or using the donor lung for research or other purposes if the one or more biomarker levels or transplant suitability score indicate that the lung will be declined for transplantation after EVLP.

A further aspect of the disclosure includes a method of selecting a donor lung for transplant, the method comprising:
i. determining in a test EVLP perfusate sample of a donor lung the level of a polypeptide of one or more biomarkers selected from the biomarkers of the invention; and
ii. comparing the level of the one or more biomarkers in the perfusate sample with a control or cut-off level or as part of an algebraic formula,
iii. selecting the donor lung for transplant according to the level(s) of the one or more biomarkers in the test EVLP perfusate sample.

In an embodiment, the method comprises:
i. obtaining one or more test EVLP perfusate samples of a perfusion solution collected during perfusion of the donor lung;
ii. measuring one or more parameter values related to a level of one or more biomarkers selected from IL-8, IL-6, sTNFR1 and sTREM-1 in the one or more test EVLP perfusate samples;
iii. optionally generating a transplant suitability score for the donor lung based on the one or more parameter values;
iv. comparing the one or more parameter values or optionally the transplant suitability score for the donor lung with a control or cut-off level;
v. selecting the donor lung for transplant according to the one or more parameter values or the transplant suitability score.

In one embodiment, the one or more parameter values comprises a concentration of the one or more biomarkers of the invention.

In one embodiment, one or more parameters values comprises a rate of biomarker production of the one or more biomarkers.

In one embodiment, the one or more parameters values comprises a ratio or fold polypeptide level of the concentration of the one or more biomarkers, wherein the ratio or fold polypeptide level is the concentration of a subsequent perfusate sample divided by the concentration of an earlier perfusate sample.

In one embodiment, the one or more parameter values comprises a concentration of the one or more biomarkers of the invention normalized to lung size (e.g. measured or predicted total lung capacity (TLC)). In one embodiment, the one or more parameter values comprise a concentration of the one or more biomarkers normalized to TLC. In one embodiment, the TLC is measured or determined based on gender and size of donor or optionally weight of the donor lung. In one embodiment, the TLC is measured or determined based on gender and height of donor.

As indicated herein an algebraic formula can be used.

Extrapolation based on perfusate levels measured during earlier time points can be useful for determining a parameter value of a concentration of a biomarker of the invention. Linear regression analysis can be used for example when assessing independent variables (e.g. polypeptide level of a biomarker and time) to predict an outcome (i.e. level of a biomarker in a future time). For example, values (e.g. 2 or more) collected from for example after 45 minutes, or after 1 hour or after 75 minutes after starting EVLP can be extrapolated to predict values expected at about 4 hours as determined by linear regression analysis. In one embodiment, the one or more parameter values comprises a concentration of the one or more biomarkers of the invention that has been extrapolated based on the perfusate levels measured during earlier time points. In some embodiments, the extrapolation is carried out with a linear regression model.

Logistic regression analysis is useful for univariate or multivariate analysis where the outcome has only a limited number of possible values. The skilled person in the art can readily recognize that logistic regression analysis is useful when the response variable is categorical in nature, such as to proceed with transplant or not. In an embodiment, ELVP outcome or patient outcome is predicted by logistic regression analysis.

It is known that the size of a subject right lung is generally larger than the size of left lung in an individual. The approximate ratio of size of right/left lung is 60:40.

In one embodiment, the one or more parameter value comprises a concentration of the one or more biomarkers of the invention that has been derived from a single right lung, single left lung, or combination thereof. In another embodiment, the one or more parameter value is adjusted based on if it is a right lung or a left lung.

In one embodiment, the one or more parameter value is used to predict PO wherein the prediction can be substratified based on donor characteristics, for example, gender, type (DBD or DCD), age, body mass index (BMI), and/or smoking history.

In some embodiments, the one or more biomarkers in the perfusate sample comprises or is IL-8.

In some embodiments, the one or more biomarkers in the perfusate sample comprises or is IL-6.

In some embodiments, the one or more biomarkers in the perfusate sample comprises or is sTNFR1.

In some embodiments, the one or more biomarkers in the perfusate sample comprises or is sTREM-1.

Different combinations of markers can be used in any of the methods described herein. In an embodiment, the one or more biomarkers is at least one of IL6 or IL-8, and optionally any one, two or three of IL-8, IL-6, sTNFR1 and sTREM-1. In an embodiment, the biomarkers are IL-6, and at least one, two or three of IL-8, sTNFR1 and sTREM-1. In an embodiment, the biomarkers are IL-8, and at least one, two or three of IL-6, sTNFR1 and sTREM-1. In an embodiment, the biomarkers are IL-6 and IL-8. In an embodiment, the biomarkers are IL-6 and sTNFR1. In an embodiment, the biomarkers are IL-6 and sTREM-1. In an embodiment, the biomarkers are IL-8 and sTNFR1. In an embodiment, the biomarkers are IL-8 and sTREM-1. In an embodiment, the biomarkers are IL-6, IL-8 and sTREM-1. In an embodiment, the biomarkers are IL-6, IL-8 and sTNFR1. In an embodiment, the biomarkers are IL-6, IL-8, sTREM-1 and sTNFR1.

In some embodiments, two biomarkers selected from IL-8, IL-6, sTNFR1 and sTREM-1 are assessed. In yet other embodiments, three biomarkers selected from IL-8, IL-6, sTNFR1 and sTREM-1 are assessed. It yet further embodiments, each of IL-8, IL-6, sTNFR1 and sTREM-1 are assessed.

In some embodiments, the method includes first collecting a test EVLP perfusate sample.

EVLP involves mechanical ventilation and pumping a nutrient (i.e. perfusion) solution such as STEEN Solution™ (or equivalent) through the blood vessels of the lungs while at the same time supplying deoxygenated gas. An aliquot of the perfusion solution that has been pumped for a selected amount can be taken while the lung continues to be on the ventilator machine or an aliquot can be taken at the end of the EVLP process.

The biomarkers described herein can be measured in test EVLP perfusate samples.

Accordingly, in some embodiments, the method first comprises:
i. inserting the donor lung into a perfusion machine;
ii. using the perfusion machine to perfuse the donor lung with an EVLP solution; wherein the one or more test EVLP perfusate samples are obtained from the EVLP solution during perfusion of the donor lung.

Typically, a lung graft receives EVLP for about 4 to 6 hours. In an embodiment, the test EVLP perfusate sample is collected during EVLP, for example at desired time points where perfusate fluid is removed from the circuit, such as after at least or at about 45 min, after at least or at about 1 hour, after at least or at about 1.5 hours, after at least or at about 2 hour, after at least or at about 2.5 hours, after at least or at about 3 hours, after at least or at about 3.5 hours, or after at least or at about 4 hours, or any time between 45 min and 6 hours, optionally any time during said interval or other intervals such as between 75 min and 2.5 hours of EVLP.

During an EVLP assessment for example, the EVLP circuit can be primed with 2000 mL of STEEN Solution™ (or equivalent). Subsequently, the circulating STEEN Solution™ within the EVLP circuit can be replenished optionally in the following manner (for example): at the end of the first hour, one-half litre of the perfusate is removed from the circuit, and one-half litre of fresh STEEN Solution™ is replaced into the circuit. After this, at the end of each subsequent hour, 0 to 500 mL of perfusate fluid can be removed from the circuit, and 500 mL of fresh Steen Solution™ can be added to the circuit. As described below, at the end of four hours of perfusion, aliquots of the test EVLP perfusate fluid were withdrawn from the perfusion circuit and frozen. The skilled person can readily determine the time and amount of perfusate fluid to be removed or replaced.

In an embodiment, the test EVLP perfusate sample is collected after at least or at about 45 min of EVLP, after at least or at about 1 hour of EVLP, after at least or at about 75 min of EVLP, after at least or at about 90 min of EVLP, after at least or at about 105 min of EVLP, after at least or at about 2 hours of EVLP, after at least or at about 3 hours of EVLP or after at least or at about 4 hours of EVLP. Test EVLP perfusate samples can also be collected at other times for example, after at least or at about 1.5 hours, after at least or at about 2.5 hours, after at least or at about 3.5 hours or after at least or at about 4.5 hours. Perfusate samples can also be collected for example at or after about 5 hours or 6 hours of EVLP. The test EVLP perfusate samples are collected within a time interval, with multiple collection within the time interval, without restriction on the intervening time between first, second and any subsequent collection of test EVLP perfusate samples. In some embodiments, the test EVLP perfusate sample is collected within a time interval, for example between any of the foregoing times. The skilled person can readily recognize that intervening time between any two perfusate sample collection can be regular or irregular, such that the intervening time between collecting any two test EVLP perfusate samples vary from another intervening time between collection any other two test EVLP perfusate samples. In some embodiments, intervening time between any two collection of EVLP perfusate samples is any time therebetween 1 min and 6 hours, optionally about 1, 2, 3, 4, 5, 6, 7, 8, 9, 10, 11, 12, 15, 20, 25, 30, 35, 40, 45, 50, 55, 60, 65, 70, 75, 80, 85, 90, 100, 110, 120, 150 min, optionally about 3, 3.5, 4, 4.5, 5, 5.5 or 6 hours. Optionally the perfusate sample is collected between 1 hour and 4 hours of EVLP, between 1 hour and 3 hours of EVLP, between 1.5 hour and 3 hours of EVLP, between 1.5 hours and 2.5 hours of EVLP or between 1 hour and 2 hours of EVLP. Other ranges can also be considered including ranges between 45 min and 6 hours.

In an embodiment, more than one test EVLP perfusate sample is collected. For example, 2 or more test EVLP perfusate samples can be collected at regular or irregular intervals. Any interval of time (e.g. every 1 min, 5 min, 10 min or 15 min or longer such as 20 min, 30 min, 45 min or 1 hour) as convenient can be assessed.

In an embodiment, a first test EVLP perfusate sample is collected after at least or at about 45 min of EVLP, and one or more subsequent test EVLP perfusate samples are collected any time therebetween 1 min and 6 hours of collecting the first perfusate sample, optionally intervening time between collecting the first and the subsequent, or any two subsequent test EVLP perfusate samples is about 1, 2, 3, 4, 5, 6, 7, 8, 9, 10, 11, 12, 15, 20, 25, 30, 35, 40, 45, 50, 55, 60, 65, 70, 75, 80, 85, 90, 100, 105, 110, 120, or 150 min, optionally about 3, 3.5, 4, 4.5, 5, 5.5 or 6 hours, optionally any time therebetween 1 min and 6 hours.

In an embodiment, the level or one or more parameter values is concentration of one or more of IL-8, IL-6, sTNFR1 or sTREM-1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-8, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-6, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of sTNFR1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of sTREM-1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-6 and IL-8, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-6 and sTNFR1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-6 and sTREM-1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-8 and sTNFR1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-8 and sTREM-1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-6, IL-8 and sTREM-1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-6, IL-8 and sTNFR1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-6, IL-8, sTREM-1 and sTNFR1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion.

As demonstrated in the Examples, the biomarkers of the invention were differentially expressed and could be used to identify donor lungs that were associated with poor PO or which were likely to be declined after EVLP. For example, the concentration of IL-8 and IL-6, as well as the rate of IL-8 and IL-6 production during EVLP was on average increased in donor lungs that were transplanted to patients that exhibited prolonged ICU stays after transplant. Similarly, IL-8 and IL-6 levels measured at 1 and 4 hours of EVLP were found to be significantly increased in donors that experienced prolonged ICU stays; this relationship was strongest in DCD donor lungs.

In an embodiment, the level or one or more parameter values determined is rate of IL-8 production.

In an embodiment, the level or one or more parameter values determined is rate of IL-6 production.

In an embodiment, the level or one or more parameter values determined is rate of sTNFR1 production.

In an embodiment, the level or one or more parameter values determined is rate of sTREM-1 production.

In another embodiment, the level or one or more parameter values is concentration of IL-8, wherein the one or more perfusate samples is a perfusate sample taken after at least or at about a time described herein, optionally after at least or at about 1 hour or 75 min of perfusion.

In an embodiment, the level or one or more parameter values is concentration of IL-8, wherein the one or more perfusate samples is a perfusate sample taken at about 4 hours of perfusion.

In another embodiment, the level or one or more parameter values is concentration of IL-6, wherein the one or more perfusate samples is a perfusate sample taken after at least or at about a time described herein, optionally after at least or at about 1 hour or 75 min of perfusion.

In an embodiment, the level or one or more parameter values is concentration of IL-6, wherein the one or more perfusate samples is a perfusate sample taken at about 4 hours of perfusion.

In another embodiment, the level or one or more parameter values is concentration of sTNFR1 wherein the one or more perfusate samples is a perfusate sample taken after at least or at about a time described herein, optionally after at least or at about 1 hour or 75 min of perfusion.

In an embodiment, the level or one or more parameter values is concentration of sTNFR1, wherein the one or more perfusate samples is a perfusate sample taken at about 4 hours of perfusion.

In another embodiment, the level or one or more parameter values is concentration of sTREM-1, wherein the one or more perfusate samples is a perfusate sample taken after at least or at about a time described herein, optionally after at least or at about 1 hour or 75 min of perfusion.

In an embodiment, the level or one or more parameter values is concentration of sTREM-1, wherein the one or more perfusate samples is a perfusate sample taken at about 4 hours of perfusion.

In an embodiment, the IL-8 polypeptide level is indicative that the donor lung optionally DCD donor lung, optionally DBD donor lung, has an increased risk of poor PO, is less or unsuitable for transplant or will be declined at the end of EVLP is an increase of at least 1.2×, 1.3×, 1.4×, 1.5×, 1.6×, 1.7×, 1.8×, 1.9×, or 2× and up to 5×, optionally any value therebetween 2× and 5×, compared to control, e.g. wherein the poor PO is >14 ICU days post-transplant. In an embodiment, the IL-8 polypeptide level is indicative that the donor lung, optionally DCD donor lung, optionally DBD donor lung, has an increased risk of poor PO, is less or unsuitable for transplant or will be declined at the end of EVLP is an increase of any value therebetween 2× and 5×, compared to control, e.g. wherein the poor PO is >14 ICU days post-transplant.

In an embodiment, the lung graft is identified as having an increased risk of poor PO, less or unsuitable for transplant or will be declined after EVLP if the level of IL-8 polypeptide in the test EVLP perfusate measured for example according to the method of Example 1, is greater than a cut-off of 1 ng/mL/HR, greater than 2 ng/mL/HR, greater than 4 ng/mL/HR, greater than 5 ng/mL/HR units or greater than 10 ng/mL/HR.

In an embodiment, the IL-6 polypeptide level is indicative that the donor lung, optionally DCD donor lung, optionally DBD donor lung, has an increased risk of poor PO is less or unsuitable for transplant or will be declined at the end of EVLP is an increase of at least 1.2×, 1.3λ, 1.4×, 1.5×, 1.6×, 1.7×, 1.8×, 1.9×, or 2×, and up to 5×, optionally any value therebetween 2× and 5×, compared to control, e.g. wherein the poor PO is >14 ICU days post-transplant. In an embodiment, the IL-6 polypeptide level is indicative that the donor lung, optionally DCD donor lung, optionally DBD donor lung has an increased risk of poor PO, is less or unsuitable for transplant or will be declined at the end of EVLP is an increase of any value therebetween 2× and 5×, compared to control, e.g. wherein the poor PO is >14 ICU days post-transplant.

In an embodiment, the lung graft is identified as having an increased risk of poor PO, as less or unsuitable for transplant or will be declined after EVLP if the level of IL-6 polypeptide in the test EVLP perfusate measured for example according to the method of Example 1, is greater than a cut-off 2 ng/mL/HR, greater than 4 ng/mL/HR, greater than 10 ng/mL/HR, greater than 20 ng/mL/HR units or greater than 50 ng/mL/HR.

In an embodiment, the sTNFR1 polypeptide level is indicative that the donor lung, optionally DCD donor lung, optionally DBD donor lung, has an increased risk of poor PO, is less or unsuitable for transplant or will be declined at the end of EVLP is an increase of at least 1.2×, 1.3×, 1.4×, 1.5×, 1.6×, 1.7×, 1.8×, 1.9×, or 2×, and up to 5×, optionally any value therebetween 2× and 5×, compared to control, e.g. poor PO is >14 ICU days post-transplant. In an embodiment, the sTNFR1 polypeptide level is indicative that the donor lung, optionally DCD donor lung, optionally DBD donor lung, has an increased risk of poor PO, is less or unsuitable for transplant or will be declined at the end of EVLP is an increase of any value therebetween 2× and 5×, compared to control, e.g. wherein the poor PO is >14 ICU days post-transplant.

In an embodiment, the lung graft is identified as having an increased risk of poor PO, as less or unsuitable for transplant or will be declined after EVLP if the level of sTNFR1 polypeptide in the test EVLP perfusate measured for example according to the method of Example 1, is greater than 1500 pg/mL of EVLP perfusate, greater than 2000 pg/mL of EVLP perfusate, greater than 3000 pg/mL of EVLP perfusate, or greater than 5000 pg/mL EVLP perfusate.

In an embodiment, the sTREM-1 polypeptide level is indicative that the donor lung, optionally DCD donor lung, optionally DBD donor lung, has an increased risk of poor PO, is less or unsuitable for transplant or will be declined at the end of EVLP is an increase of at least 1.2×, 1.3×, 1.4×, 1.5×, 1.6×, 1.7×, 1.8×, 1.9×, or 2×, and up to 5×, compared to control, e.g. poor PO is >14 ICU days post-transplant. In an embodiment, the sTREM-1 polypeptide level is indicative that the donor lung optionally DCD donor lung, optionally DBD donor lung, has an increased risk of poor PO, is less or unsuitable for transplant or will be declined at the end of EVLP is an increase of any value therebetween 2× and 5×, compared to control, e.g. wherein the poor PO is >14 ICU days post-transplant.

In an embodiment, the lung graft is identified having an increased risk of poor PO, as less or unsuitable for transplant or will be declined after EVLP if the level of sTREM-1 polypeptide in the test EVLP perfusate measured for example according to the method of Example 1, is greater than 500 pg/mL of EVLP perfusate, greater than 1000 pg/mL of EVLP perfusate, greater than 2000 pg/mL of EVLP perfusate or greater than 4000 pg/mL EVLP perfusate.

Example 1 also demonstrates that a number of biomarker parameters could be combined and which were better than the best univariate model at predicting patient outcome such as long ICU stay (e.g. greater than 2 weeks). In one embodiment, the combination of biomarkers and/or the one or more parameters are at least two parameters selected from any of the foregoing: 1) IL-6 concentration after at least or at about 1 hour of perfusion and about 4 hours of perfusion or any time therebetween, optionally between 1 hour and 3 hours or between 1.5 hours and 2.5 hours of perfusion; 2) IL-8 concentration at about 2 hours, about 3 hours or about 4 hours of perfusion and sTNFR1 concentration at about 2 hours, about 3 hours or about 4 hours of perfusion; 3) IL-6 concentration after at least or at about 1 hour of perfusion and about 4 hours of perfusion optionally between 1 hour and 3 hours or between 1.5 hours and 2.5 hours of perfusion and sTREM-1 after at least or at about 1 hour or about 2 hours of perfusion or any time therebetween; 4) IL-6 concentration after at least or at about 1 hour or about 2 hours of perfusion and sTREM-1 concentration after at least or at about 1 hour or about 2 hours of perfusion and IL-8 concentration at about 2 hours, about 3 hours or 4 hours of perfusion, 5) sTNFR1 concentration after at least or at about 1 hour of perfusion and about 4 hours of perfusion optionally between 1 hour and 3 hours or between 1.5 hours and 2.5 hours of perfusion and IL-8 concentration at about 2 hours, about 3 hours or about 4 hours of perfusion, 6) sTREM-1 concentration after at least or at about 1 hour or about 2 hours of perfusion or any time therebetween and IL-8 concentration at about 2 hours, about 3 hours or about 4 hours of perfusion and sTNFR1 concentration at about 2 hours, about 3 hours or about 4 hours of perfusion, and 7) IL-6 concentration at about 1 hour of perfusion and about 4 hours of perfusion, optionally between 1 hour and 3 hours or between 1.5 hours and 2.5 hours of perfusion and sTREM-1 after at least or at about 1 hour or about 2 hours of perfusion or any time therebetween and sTNFR1 at about 2 hours, about 3 hours or about 4 hours of perfusion.

Example 1 also identified a number of combinations that were better than a univariate model at predicting whether a lung would be declined at the end of EVLP.

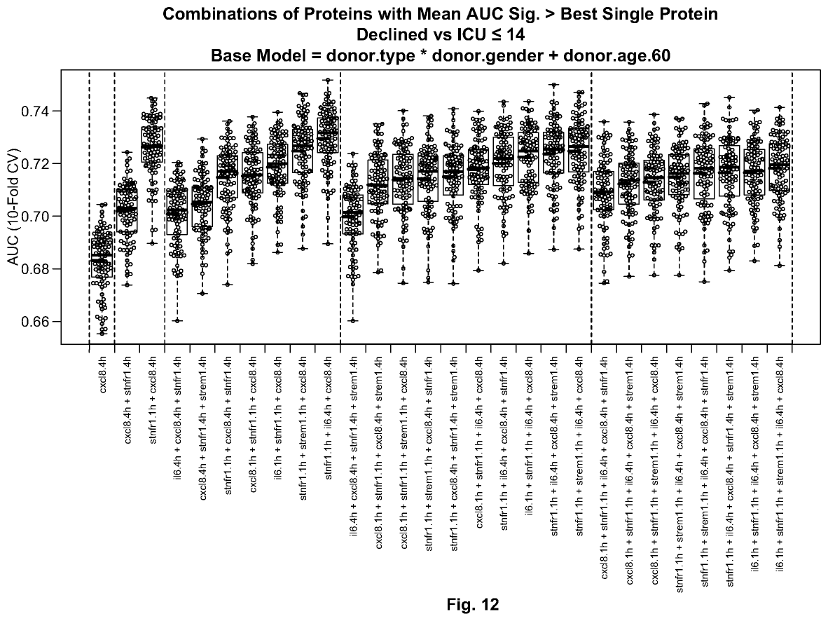

Accordingly in an embodiment, the combination and/or one or more parameters are at least two parameters selected from the combinations listed in FIG. 12. In an embodiment, combinations include biomarkers of one or more of A) IL-6; IL-8; sTNFR1; sTREM-1; IL-6 and IL-8; sTNFR1 and sTREM-1; IL-6 and sTNFR1; IL-6 and sTREM-1; IL-8 and sTNFR1; IL-8 and sTREM-1; IL-6, IL-8 and sTNFR1; IL-6, IL-8 and sTREM-1; and IL-6, IL-8, sTNFR1 and sTREM-1; combining with one or more of EVLP perfusate sample collected at B) any time therebetween 45 min and 6 hours of EVLP, optionally after at least or at about 45 min of EVLP, after at least or at about 1 hour of EVLP, after at least or at about 75 min of EVLP, after at least or at about 1.5 hours of EVLP, after at least or at about 105 min of EVLP, after at least or at about 2 hours of EVLP, after at least or at about 2.5 hours of EVLP, after at least or at about 3 hours of EVLP, after at least or at about 3.5 hours of EVLP, after at least or at about 4 hours of EVLP, after at least or at about 4.5 hours of EVLP, after at least or at about 5 hours of EVLP, after at least or at about 5.5 hours of EVLP, after at least or at about 6 hours of EVLP, optionally intervening time between collecting any two test EVLP perfusate samples is about 1, 2, 3, 4, 5, 6, 7, 8, 9, 10, 11, 12, 15, 20, 25, 30, 35, 40, 45, 50, 55, 60, 65, 70, 75, 80, 85, 90, 100, 110, 120, 150 min, optionally about 3, 3.5, 4, 4.5, 5, 5.5 or 6 hours, optionally any time therebetween 1 min and 6 hours.

In an embodiment, the parameters are at least one of the combination selected from: 1) IL-8 concentration after at least or at about 4 hours of perfusion and sTNFR1 concentration after at least or at about 4 hours of perfusion; 2) sTNFR1 concentration after at least or at about 1 hour of perfusion and IL-8 concentration after at least or at about 4 hours of perfusion; 3) IL-6 concentration after at least or at about 4 hours of perfusion, IL-8 concentration after at least or at about 4 hours of perfusion, and sTNFR1 concentration after at least or at about 4 hours of perfusion; 4) IL-8 concentration after at least or at about 4 hours of perfusion, sTNFR1 concentration after at least or at about 4 hours of perfusion, and sTREM-1 concentration after at least or at about 4 hours of perfusion; 5) sTNFR1 concentration after at least or at about 1 hour of perfusion, IL-8 concentration after at least or at about 4 hours of perfusion, and sTNFR1 concentration after at least or at about 4 hours of perfusion; 6) IL-8 concentration after at least or at about 1 hour of perfusion, sTNFR1 concentration after at least or at about 1 hour of perfusion, and IL-8 concentration after at least or at about 4 hours of perfusion; 7) IL-6 concentration after at least or at about 1 hour of perfusion, sTNFR1 concentration after at least or at about 1 hour of perfusion, and IL-8 concentration after at least or at about 4 hours of perfusion; 8) sTNFR1 concentration after at least or at about 1 hour of perfusion, sTREM-1 concentration after at least or at about 1 hour of perfusion, and IL-8 concentration after at least or at about 4 hours of perfusion; 9) sTNFR1 concentration after at least or at about 1 hour of perfusion, IL-6 concentration after at least or at about 4 hours of perfusion, and IL-8 concentration after at least or at about 4 hours of perfusion; 10) IL-6 concentration after at least or at about 4 hours of perfusion, IL-8 concentration after at least or at about 4 hours of perfusion, sTNFR1 concentration after at least or at about 4 hours of perfusion, and sTREM-1 concentration after at least or at about 4 hours of perfusion; 11) IL-8 concentration after at least or at about 1 hour of perfusion, sTNFR1 concentration after at least or at about 1 hour of perfusion, IL-8 concentration after at least or at about 4 hours of perfusion, and sTREM-1 concentration after at least or at about 4 hours of perfusion; 12) IL-8 concentration after at least or at about 1 hour of perfusion, sTNFR1 concentration after at least or at about 1 hour of perfusion, sTREM-1 concentration after at least or at about 1 hour of perfusion, and IL-8 concentration after at least or at about 4 hours of perfusion; 13) sTNFR1 concentration after at least or at about 1 hour of perfusion, sTREM-1 concentration after at least or at about 1 hour of perfusion, IL-8 concentration after at least or at about 4 hours of perfusion, and sTNFR1 concentration after at least or at about 4 hours of perfusion; 14) sTNFR1 concentration after at least or at about 1 hour of perfusion, IL-8 concentration after at least or at about 4 hours of perfusion, sTNFR1 concentration after at least or at about 4 hours of perfusion, and sTREM-1 concentration after at least or at about 4 hours of perfusion; 15) IL-8 concentration after at least or at about 1 hour of perfusion, sTNFR1 concentration after at least or at about 1 hour of perfusion, IL-6 concentration after at least or at about 4 hours of perfusion, and IL-8 concentration after at least or at about 4 hours of perfusion; 16) sTNFR1 concentration after at least or at about 1 hour of perfusion, IL-6 concentration after at least or at about 4 hours of perfusion, IL-8 concentration after at least or at about 4 hours of perfusion, and sTNFR1 concentration after at least or at about 4 hours of perfusion; 17) IL-6 concentration after at least or at about 1 hour of perfusion, sTNFR1 concentration after at least or at about 1 hour of perfusion, IL-6 concentration after at least or at about 4 hours of perfusion, and IL-8 concentration after at least or at about 4 hours of perfusion; 18) sTNFR1 concentration after at least or at about 1 hour of perfusion, IL-6 concentration after at least or at about 4 hours of perfusion, IL-8 concentration after at least or at about 4 hours of perfusion, and sTREM-1 concentration after at least or at about 4 hours of perfusion; 19) sTNFR1 concentration after at least or at about 1 hour of perfusion, sTREM-1 concentration after at least or at about 1 hour of perfusion, IL-6 concentration after at least or at about 4 hours of perfusion, and IL-8 concentration after at least or at about 4 hours of perfusion; 20) IL-8 concentration after at least or at about 1 hour of perfusion, sTNFR1 concentration after at least or at about 1 hour of perfusion, IL-6 concentration after at least or at about 4 hours of perfusion, IL-8 concentration after at least or at about 4 hours of perfusion, and sTNFR1 concentration after at least or at about 4 hours of perfusion; 21) IL-8 concentration after at least or at about 1 hour of perfusion, sTNFR1 concentration after at least or at about 1 hour of perfusion, IL-6 concentration after at least or at about 4 hours of perfusion, IL-8 concentration after at least or at about 4 hours of perfusion, and sTREM-1 concentration after at least or at about 4 hours of perfusion; 22) IL-8 concentration after at least or at about 1 hour of perfusion, sTNFR1 concentration after at least or at about 1 hour of perfusion, sTREM-1 concentration after at least or at about 1 hour of perfusion, IL-6 concentration after at least or at about 4 hours of perfusion, and IL-8 concentration after at least or at about 4 hours of perfusion; 23) sTNFR1 concentration after at least or at about 1 hour of perfusion, sTREM-1 concentration after at least or at about 1 hour of perfusion, IL-6 concentration after at least or at about 4 hours of perfusion, IL-8 concentration after at least or at about 4 hours of perfusion, and sTREM-1 concentration after at least or at about 4 hours of perfusion; 24) sTNFR1 concentration after at least or at about 1 hour of perfusion, sTREM-1 concentration after at least or at about 1 hour of perfusion, IL-6 concentration after at least or at about 4 hours of perfusion, IL-8 concentration after at least or at about 4 hours of perfusion, and sTNFR1 concentration after at least or at about 4 hours of perfusion; 25) sTNFR1 concentration after at least or at about 1 hour of perfusion, IL-6 concentration after at least or at about 4 hours of perfusion, IL-8 concentration after at least or at about 4 hours of perfusion, sTNFR1 concentration after at least or at about 4 hours of perfusion, and sTREM-1 concentration after at least or at about 4 hours of perfusion; 26) IL-6 concentration after at least or at about 1 hour of perfusion, sTNFR1 concentration after at least or at about 1 hour of perfusion, IL-6 concentration after at least or at about 4 hours of perfusion, IL-8 concentration after at least or at about 4 hours of perfusion, and sTREM-1 concentration after at least or at about 4 hours of perfusion; and 27) IL-6 concentration after at least or at about 1 hour of perfusion, sTNFR1 concentration after at least or at about 1 hour of perfusion, sTREM-1 concentration after at least or at about 1 hour of perfusion, IL-6 concentration after at least or at about 4 hours of perfusion, and IL-8 concentration after at least or at about 4 hours of perfusion.

The levels of the polypeptide biomarkers can be detected using a number of methods known in the art. For example, the methods can include immunoassays such as ELISA and multiplex assays including Luminex® based assays, flow cytometry, Western blots, and immunoprecipitation followed by SDS-PAGE immunocytochemistry. Protein microarrays are also useful.

In an embodiment, the levels of two or more of IL-8, IL-6, sTNFR1 and sTREM-1 polypeptides are detected for example using a Luminex® assay.

In an embodiment, the level one or more IL-8, IL-6, sTNFR1 and sTREM-1 polypeptide is detected or determined by immunohistochemistry (IHC), Luminex® based assays, Western blots, ELISA, immunofluorescence, radioimmunoassay, dot blotting, FACS, protein microarray, immunoprecipitation followed by SDS-PAGE, immunocytochemistry, Simple Plex assay or Mass Spectrometry.

An at least 1.2 fold difference means for example that the level of the biomarker in the test perfusate sample is at least 120% the level in a control comparator perfusate sample or derived value.

In an embodiment, the method involves comparing to a cut-off. For example each marker will have a different cut-off depending on statistical calculations and/or desired test sensitivity and/or specificity. Where more than one biomarker is assessed, a composite score can be determined.

In an embodiment, the poor outcome lung grafts are characterized as being unsuitable for clinical transplantation after EVLP or, in the recipient after transplantation, inducing death from graft-related causes within 30 days, PGD3 or requiring extracorporeal life support/ECMO, or prolonged hospital/ICU/mechanical ventilation days.

As demonstrated in the Examples, the clinical measures of patient outcome (e.g. ICU length of stay, total hospital length of stay, etc.) were found to be significantly correlated with each other (e.g. Days on a ventilator with total ICU length of stay); this correlation between measures of outcome was also found to exist for a patient's APACHE score.

The biomarker levels can for example be measured using various immunological and/or proteomic assays. For example, the polypeptide level of a biomarker of the invention can be measured using a Simple Western or Simple Plex (Protein Simple, San Jose, Calif., USA). In some embodiments, the perfusate is diluted prior to measuring.

### III. Immunoassays and Kits

An aspect of the disclosure also includes kits containing antibodies for the detection of the biomarkers of the invention that are used to measure the biomarker levels, i.e. polypeptide levels.

In an embodiment the kit comprises an immunoassay for one or more of biomarkers of the invention. Each kit comprises at least one detection antibody specific for a biomarker of the invention. For example, the antibody may be in the form of antibody coupled beads such as antibody coupled magnetic beads, or labelled antibodies, optionally comprised in a cartridge. In an embodiment, the kit further comprises one or more of a 96-well plate, a cartridge comprising one or more antibodies, standards, assay buffer, wash buffer, sample diluent, standard diluent, detection antibody diluent, streptavidin-PE, a filter plate and sealing tape. In an embodiment the kit comprises detection antibodies or assays for detecting two or more biomarkers of the invention e.g. two or more of IL-8, IL-6, sTNFR1 and sTREM-1. In an embodiment, the kit comprises detection antibodies or assays for detecting IL-6 and IL-8. In another embodiment, the kit comprises detection antibodies or assays for detecting any one of IL-8 and IL-6 and one or more of IL-8, IL-6, sTNFR1 and sTREM-1 are assessed. In another embodiment, the kit comprises detection antibodies or assays for detecting IL-8 and at least one, two or three IL-6, sTNFR1 and sTREM-1. In another embodiment, the kit comprises detection antibodies or assays for detecting IL-6 and at least one, two or three IL-8, sTNFR1 and sTREM-1. In another embodiment, the kit comprises detection antibodies or assays for detecting IL-6 and sTNFR1. In another embodiment, the kit comprises detection antibodies or assays for detecting IL-6 and sTREM-1. In another embodiment, the kit comprises detection antibodies or assays for detecting IL-8 and sTNFR1. In another embodiment, the kit comprises detection antibodies or assays for detecting IL-8 and sTREM-1. In another embodiment, the kit comprises detection antibodies or assays for detecting IL-6, IL-8 and sTREM-1. In another embodiment, the kit comprises detection antibodies or assays for detecting IL-6, IL-8 and sTNFR1. In another embodiment, the kit comprises detection antibodies or assays for detecting IL-6, IL-8, sTREM-1 and sTNFR1.

In an embodiment, the kits are for use for a method described herein.

In an embodiment, the kit further comprises detection agents for other known lung graft outcome markers.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

EVLP perfusate samples were collected during the course of an EVLP procedure. These perfusate samples were then analyzed for the polypeptide levels of IL-6, IL-8, sTNFR-1, and sTREM-1. The polypeptide levels (either absolute concentration, rate of increase or fold-increase) were included in a mathematical calculation that may include other known clinical variables (e.g. donor type). The output of this calculation provides a predictive score of whether or not the lung is likely to be declined at the end of EVLP and therefore it is prudent to stop EVLP at that time or, if transplanted, the patient that receives the lung is likely to experience a prolonged ICU stay. At that point the surgeon can then decide the relative risk to the patient and decide whether or not to carry out the transplant.

### Methods

Patient Selection:

Approximately 210 cases of double lung transplantation using EVLP were identified. 110 donor organs were sourced from donation after brain death (DBD) and 100 cases arose from donation after cardiac death (DCD).

Perfusate Sample:

Approximately 1 mL of Perfusate solution was removed from the EVLP circuit 1 and 4 hours after the procedure began. Perfusate samples were snap frozen and used in the following experiments.

Biomarker Quantification:

Perfusate samples were diluted as per manufacturer's instructions in calibrator diluent. A set of standards for the generation of a standard curve was prepared concurrently (Protein Simple). Each plate was prepared according to the manufacturer's protocol (Protein Simple). Each plate was then run and read on the Simple Plex System (Protein Simple), which was set up and calibrated as per the manufacturer's instructions.

### Statistical Analysis:

Regression analysis was carried out using Prism 7 (Graph Pad), SPSS

Statistics (IBM), or R software environment. Development of the predictive models was performed using logistic regression analysis on the measured EVLP biomarkers and was carried out in all combinations of measured markers. Cross-validation of each model was carried out using 100 rounds of 10-fold cross-validation with stratification. For all statistical calculations, a p-value of less than 0.05 was considered statistically significant.

### Results

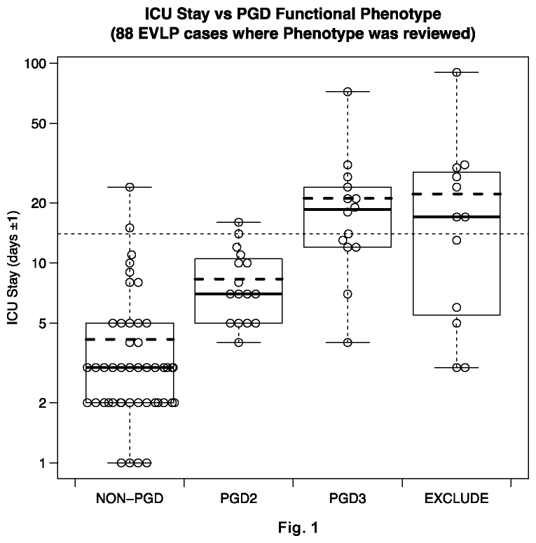

As shown in FIG. 1, a significant difference was seen in the number of days that a patient would spend in the ICU (ICU length of stay) between the various PGD classifications (NON-PGD, PGD2, PGD3). Some cases were ‘excluded’ from the analysis following clinical review.

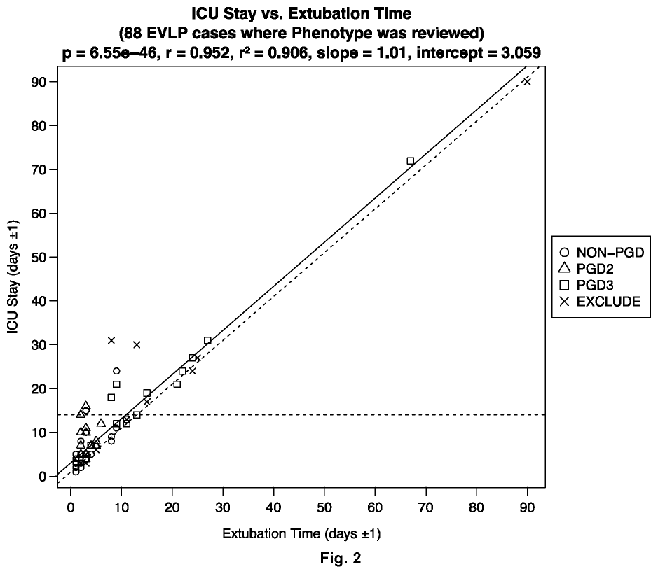

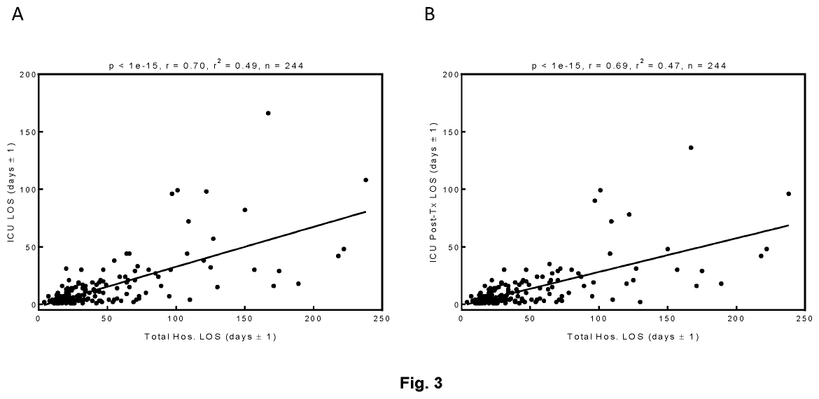

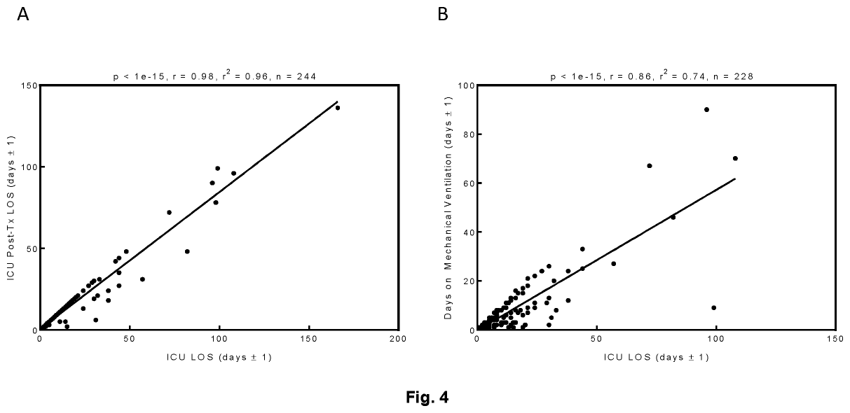

In FIG. 2 through 4, significant correlation was observed for the comparisons of various measures of patient outcome (e.g. ICU length of stay (ICU LOS), hospital length of stay, days on a ventilator). A similar relationship was also found for days on a ventilator with total transplant related length of stay as well as a patient's APACHE score with ventilator days, ICU LOS, post-transplant LOS, and total LOS.

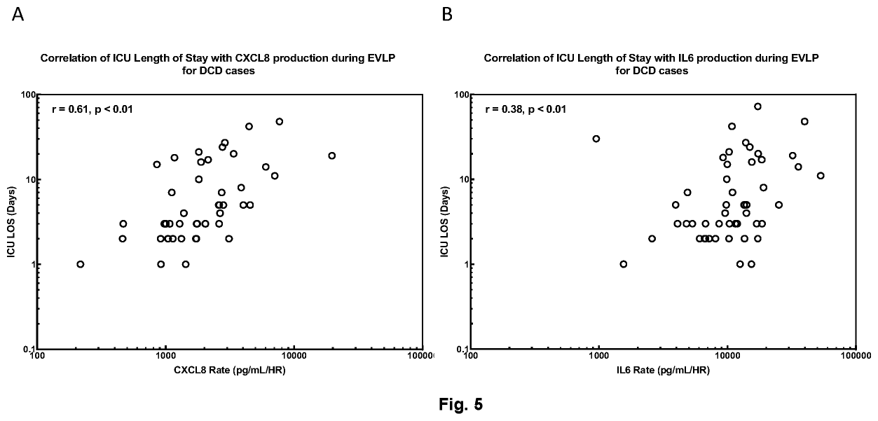

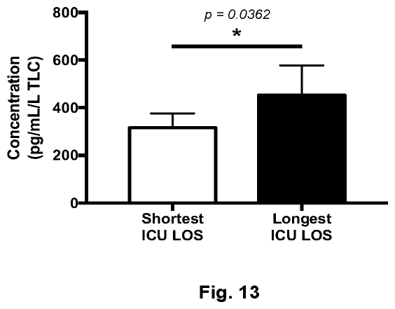

FIG. 5 depicts the significant, positive, relationship between the rate of IL-8 and IL-6 production and length of ICU stay. Similar trends were observed at the 1 and 4-hour point of EVLP for IL-8 and IL-6 and this was particularly strong in DCD donors. FIG. 13 depicts the significant, positive, relationship between sTNFR1 1-hour concentration and length of ICU stay.

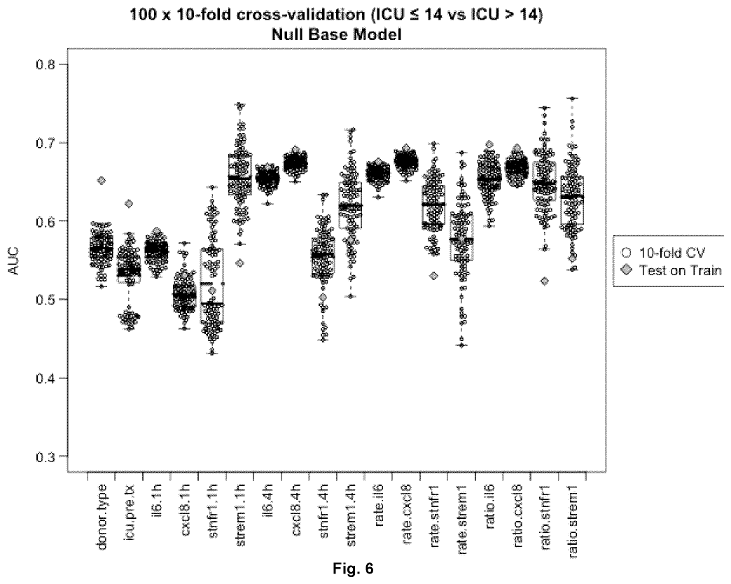

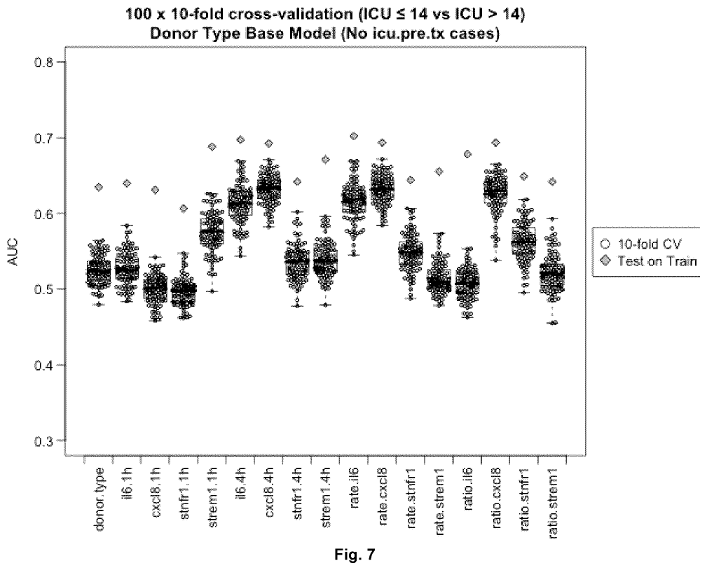

FIGS. 6 and 7 show the logistic regression results for the univariate models that predict ICU length of stay greater than 14 days. In FIG. 6 each biomarker is compared to the Null hypothesis (base model) and in FIG. 7 the comparison is made to the best clinical model (e.g. donor type). Each diamond represents the AUC for the test on train model and the Box and Whisker plots (open circles) show the cross-validation results. IL-6 and IL-8 polypeptide levels at 4 hours were found to be the most predictive and significantly more predictive than the best clinical model.

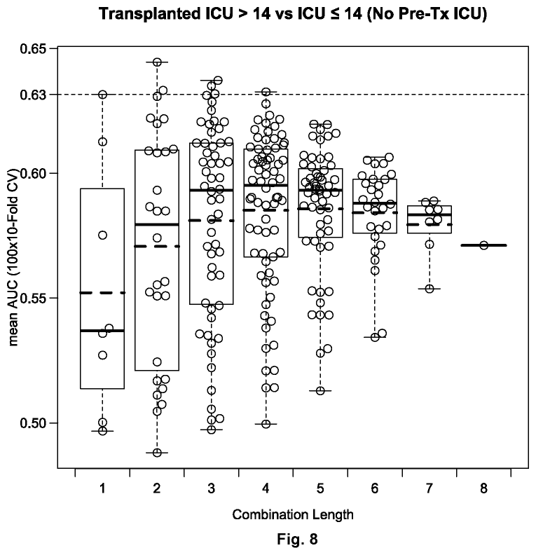

Shown in FIG. 8 are the results of the multivariate analysis for the prediction of ICU stays greater than 14 days. AUCs for the various combinations of 1 to 8 biomarker parameters (i.e. concentration at 1 hour of IL-6, IL-8, sTNFR1 and/or sTREM-1 and/or concentration at 4 hours IL-6, IL-8, sTNFR1 and/or sTREM-1) are shown as open circles with Box and Whisker plots. The AUC of the best univariate model (IL-8 at 4 hours) is shown as a dashed line. Biomarker combinations that improved the model were: IL-6 (1 and 4 hours), IL-8 and sTNFR1 (4 hours), IL-6 (1 and 4 hours) and sTREM-1 (1 hour), IL-6 (1 hour) and sTREM-1 (1 hour) and IL-8 (4 hours), sTNFR1 (1 and 4 hours) and IL-8 (4 hours), sTREM-1 (1 hour) and IL-8 (4 hours) and sTNFR1 (4 hours), IL-6 (1 and 4 hours) and sTREM-1 (1 hour) and sTNFR1 (4 hours).

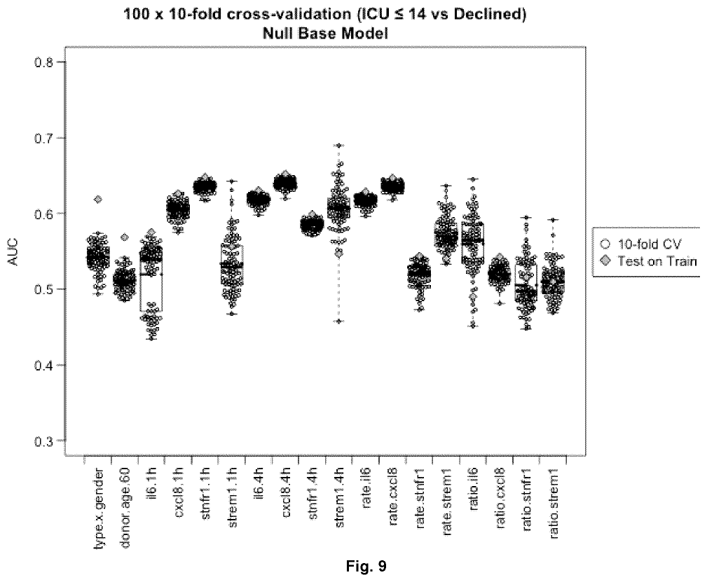

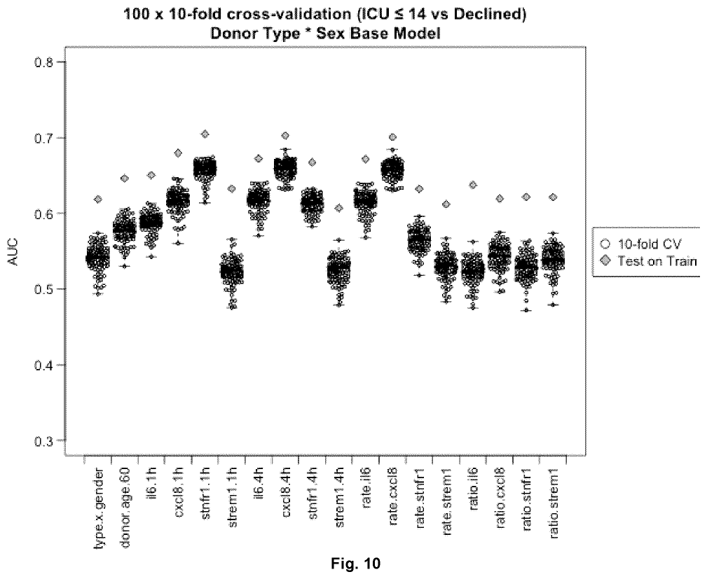

FIGS. 9 and 10 show the logistic regression results for the univariate models that predict lungs that would be declined on EVLP. In FIG. 9 each biomarker is compared to the Null hypothesis (base model) and in FIG. 10 the comparison is made to the best clinical model (e.g. donor type and donor gender). Each diamond represents the AUC for the test on train model and the Box and Whisker plots (open circles) show the cross-validation results. IL-8 polypeptide levels at 4 hours were found to be the most predictive and significantly more predictive than the best clinical model.

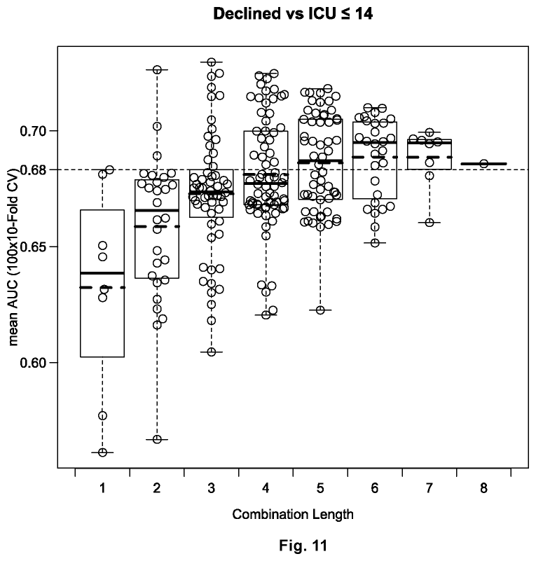

Shown in FIG. 11 are the results of the multivariate analysis for the prediction of declined EVLP cases. AUCs for the various combinations of 1 to 8 biomarkers parameters (i.e. concentration at 1 hour of IL-6, IL-8, sTNFR1 and/or sTREM-1 and/or concentration at 4 hours IL-6, IL-8, sTNFR1 and/or sTREM-1) are shown as open circles with Box and Whisker plots. The AUC of the best univariate model (IL-8 at 4 hours) is shown as a dashed line. Biomarker combinations that were significantly better than the base clinical model and univariate marker are shown and listed in FIG. 12.

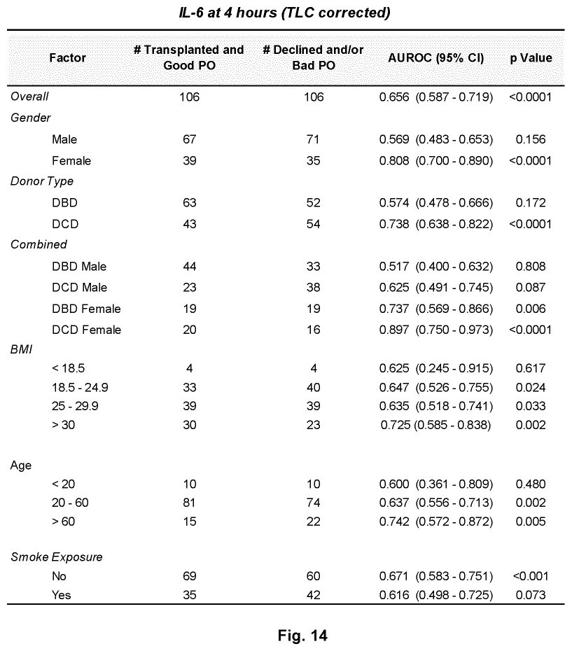

FIG. 14 shows that the performance (area under the ROC curve (AUROC)) for predicting PO can be substratified for various donor characteristics (e.g. gender, donor type (DCD or DBD), age, body mass index, smoking history).

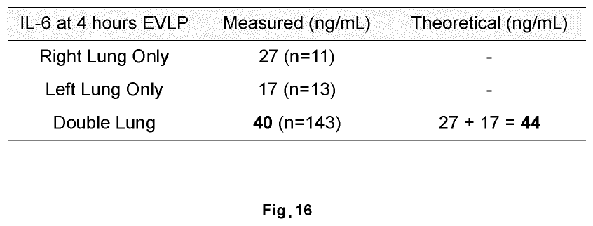

FIG. 16 shows that a theoretical double lung (sum of a single left and single right lung with good PO) is comparable to the observed double lungs transplanted with good PO.

### Example 2

Patient Selection:

40 cases of double lung transplantation using EVLP were identified. 26 cases were transplanted with an outcome of PGD0/1 and 14 cases were transplanted with an outcome of PGD3. 17 donor organs were sourced from donation after brain death (DBD) and 23 cases arose from donation after cardiac death (DCD).

Perfusate Sample:

Approximately 1 mL of Perfusate solution was removed from the EVLP circuit 4 hours after the procedure began. Perfusate samples were snap frozen and used in the following experiments.

Biomarker Quantification:

Perfusate samples were diluted as per manufacturer's instructions in calibrator diluent. A set of standards for the generation of a standard curve was prepared concurrently (Luminex). Each plate was prepared according to the manufacturer's protocol (Luminex). Each plate was then run and read on the MagPix system (Luminex), which was set up and calibrated as per the manufacturer's instructions.

Statistical Analysis:

Differences in biomarker polypeptide levels for transplant outcome were determined for PGD0/1 and PGD3. Statistical calculations and analysis were carried out using Prism 7 (GraphPad) and SPSS Statistics (IBM) software. For all statistical calculations, a p-value of less than 0.05 was considered statistically significant.

### Results

A significant difference was seen in the polypeptide level of each biomarker between lung transplant outcomes that were characterized as PGD0/1 or PGD3 for each of IL-6, IL-8, sTNFR1 and sTREM-1. For IL-8, the increase in the average level measured in patients identified as PGD3 compared to PGD0 was about 28% when all transplants were considered. The difference was even greater for DCD transplants. For IL-6, the increase in the average level measured in patients identified as PGD3 compared to PGD0 was about 25% when all transplants were considered. Again, the difference was even greater for DCD transplants. For sTREM-1, the average concentration in pg/mL was about 3800 pg/mL for patients developing PGD0 and about 4500 pg/mL for patients developing PGD3. Again the difference was even greater for DCDs (about 4700 p/mL). Differences were also seen for sTNFR1.

### Example 3

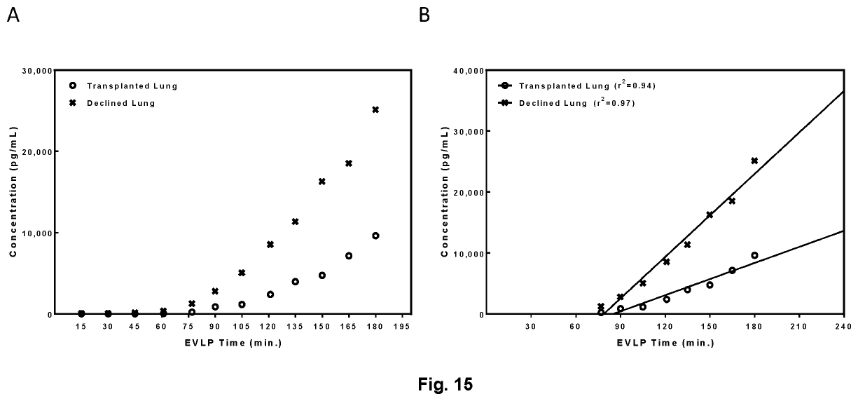

FIG. 15 shows EVLP perfusate samples that were collected during the course of an EVLP procedure similar to the one described for example in Example 1 or 2, but collected every 15 minutes for up to 3 hours. These perfusate samples were analyzed for the polypeptide levels of one or more biomarkers described for example in Example 1 and 2.

FIG. 15 shows the different biomarker levels of declined and transplanted lungs from 0 through 3 hours (180 minutes). FIG. 15 also shows that the biomarker levels collected during 15 minute intervals are useful to generate a mathematical model that can then be used to predict future biomarker levels (e.g. 4 hours (240 minutes)). The person skilled in the art understands that collection time between any two perfusate samples can vary from 1 min to 6 hours, and intervening times for collecting perfusate samples can vary between any two perfusate samples.

### CITATIONS FOR REFERENCES REFERRED TO IN THE SPECIFICATION

1. M. Cypel, S. Keshavjee, The clinical potential of ex vivo lung perfusion. Expert Rev Respir Med 6, 27-35 (2012).
2. M. Cypel, M. Rubacha, J. Yeung, S. Hirayama, K. Torbicki, M. Madonik, S. Fischer, D. Hwang, A. Pierre, T. K. Waddell, M. de Perrot, M. Liu, S. Keshavjee, Normothermic ex vivo perfusion prevents lung injury compared to extended cold preservation for transplantation. Am J Transplant 9, 2262-2269 (2009).
3. M. Cypel, J. C. Yeung, S. Hirayama, M. Rubacha, S. Fischer, M. Anraku, M. Sato, S. Harwood, A. Pierre, T. K. Waddell, M. de Perrot, M. Liu, S. Keshavjee, Technique for prolonged normothermic ex vivo lung perfusion. J Heart Lung Transplant 27, 1319-1325 (2008).
4. M. Cypel, J. C. Yeung, M. Liu, M. Anraku, F. Chen, W. Karolak, M. Sato, J. Laratta, S. Azad, M. Madonik, C. W. Chow, C. Chaparro, M. Hutcheon, L. G. Singer, A. S. Slutsky, K. Yasufuku, M. de Perrot, A. F. Pierre, T. K. Waddell, S. Keshavjee, Normothermic ex vivo lung perfusion in clinical lung transplantation. N Engl J Med 364, 1431-1440 (2011).
5. J. C. Yeung, M. Cypel, E. Massad, S. Keshavjee, Ex vivo lung perfusion and reconditioning. Multimed Man Cardiothorac Surg 2011, mmcts 2009 004242 (2011).
6. M. Cypel, J. C. Yeung, S. Keshavjee, Novel approaches to expanding the lung donor pool: donation after cardiac death and ex vivo conditioning. Clin Chest Med 32, 233-244 (2011).
7. M. K. Hsin, I. Iskender, D. Nakajima, M. Chen, H. Kim, P. R. dos Santos, J. Sakamoto, J. Lee, K. Hashimoto, C. Harmantas, D. Hwang, T. Waddell, M. Liu, S. Keshavjee, M. Cypel, Extension of donor lung preservation with hypothermic storage after normothermic ex vivo lung perfusion. J Heart Lung Transplant 35, 130-136 (2016).
8. T. N. Machuca, O. Mercier, S. Collaud, J. Tikkanen, T. Krueger, J. C. Yeung, M. Chen, S. Azad, L. Singer, K. Yasufuku, M. de Perrot, A. Pierre, T. K. Waddell, S. Keshavjee, M. Cypel, Lung transplantation with donation after circulatory determination of death donors and the impact of ex vivo lung perfusion. Am J Transplant 15, 993-1002 (2015).
9. T. N. Machuca, M. Cypel, J. C. Yeung, R. Bonato, R. Zamel, M. Chen, S. Azad, M. K. Hsin, T. Saito, Z. Guan, T. K. Waddell, M. Liu, S. Keshavjee, Protein expression profiling predicts graft performance in clinical ex vivo lung perfusion. Ann Surg 261, 591-597 (2015).

1. A method for the screening, diagnosing, or detecting of outcome/risk as it relates to donor lungs, comprising:
i. determining, in one or more test EVLP perfusate samples of a donor lung, the level of polypeptide and/or one or more parameter values related to said polypeptide level of one or more biomarkers selected from IL-6 and IL-8 and optionally one or more of sTREM-1 and sTNFR1; and
ii. a) comparing the level of the one or more biomarkers or the one or more parameter values in the one or more perfusate samples with a control or cut-off level, wherein the differential level is indicative of outcome/risk of after transplant; or
b) using the polypeptide level and/or the one or more parameter values of several of the biomarkers in combination, as part of an algebraic calculation of outcome/risk after transplant.

2. The method of claim 1, wherein the outcome/risk is risk of a negative post-lung transplant patient outcome (PO), or the outcome/risk is determined to be sufficient to discontinue EVLP, and optionally wherein the PO is selected from extended intensive care unit (ICU) length of stay, extended time on ventilator and/or extended post-transplant hospital stay.

3. (canceled)

4. (canceled)

5. The method of claim 1 for predicting a patient outcome (PO) variable for a lung transplant recipient after transplant, wherein step i. comprises:
obtaining one or more test EVLP perfusate samples of a perfusion solution collected during perfusion of a donor lung;
measuring in the one or more test EVLP perfusate samples one or more parameter values related to a polypeptide level of one or more biomarkers selected from IL-8 and IL-6, and optionally one or more of sTNFR1 and sTREM-1;
optionally generating a PO variable score for the donor lung based on the one or more parameter values; and
step ii. comprises:
comparing one or more parameter values or optionally the PO score for the donor lung with a control or cut-off level, wherein the PO variable score is indicative of a PO variable after transplant.

6. The method of claim 5, wherein the outcome/risk or PO variable is selected from ICU length of stay, post-transplant hospital length of stay, number of days on a ventilator, APACHE score and post graft dysfunction (PGD) grade, optionally PGD0/1 or PGD3.

7.-11. (canceled)

12. The method of claim 1, further comprising i) selecting said donor lung for transplant if the outcome/risk or PO variable is acceptable or below the cutoff value, preparing said donor lung for transplant and/or transplanting said donor lung into a suitable recipient, or ii) discarding the donor lung and/or using the donor lung for research or other purposes if the outcome/risk or PO variable is unacceptable or above the cut-off value.

13. (canceled)

14. A method for the early detection of donor lungs that will be declined at the end of the EVLP process comprising:
a) obtaining one or more test EVLP perfusate samples of a perfusion EVLP solution collected during perfusion of the donor lung;
b) determining one or more parameter values related to a level of one or more biomarkers selected from IL-8 and IL-6, and optionally one or more of sTNFR1 and sTREM-1, in the one or more test EVLP perfusate samples;
c) optionally generating a transplant suitability score for the donor lung based on the one or more parameter values;
d) comparing the one or more parameter values or optionally the transplant suitability score for the donor lung with a control or cut-off level, and
e) continuing perfusion if the one or more parameter values or transplant suitability score indicates that the donor lung is suitable for transplantation and discontinuing perfusion if the one or more parameter values or the transplant suitability score indicates that the donor lung will be declined for transplantation.

15. The method of claim 14, further comprising discarding the donor lung and/or using the donor lung for research or other purposes if one or more parameter values or transplant suitability score indicates that the lung will be declined for transplantation after EVLP.

16. A method of selecting a candidate donor lung for transplant, the method comprising:
a) obtaining one or more test EVLP perfusate samples of a perfusion solution collected during perfusion of the donor lung;
b) measuring one or more parameter values related to a level of one or more biomarkers selected from IL-8, IL-6, sTNFR1 and sTREM-1 in the one or more test EVLP perfusate samples;
c) optionally generating a transplant suitability score for the donor lung based on the one or more parameter values;
d) comparing the one or more parameter values or optionally the transplant suitability score for the donor lung with a control or cut-off level;
e) selecting the donor lung for transplant according to the one or more parameter values or the transplant suitability score.

17. The method of claim 1, wherein the method first comprises:
a) inserting the donor lung into a perfusion machine;
b) using the perfusion machine to perfuse the donor lung with an EVLP solution; wherein the one or more test EVLP perfusate samples are obtained from the EVLP solution during perfusion of the donor lung.

18. The method of claim 1, wherein the one or more test EVLP perfusate samples are collected after at least or at about 45 min of EVLP, after at least or at about 1 hour of EVLP, after at least or at about 75 min of EVLP, after at least or at about 1.5 hours of EVLP, after at least or at about 2 hours of EVLP, after at least or at about 2.5 hours of EVLP, after at least or at about 3 hours of EVLP, after at least or at about 3.5 hours of EVLP and/or after at least or at about 4 hours of EVLP, preferably after at least or at about 45 min and/or about 4 hours, or any time therebetween, optionally between 1 hour and 4 hours of EVLP, between 1 hour and 3 hours of EVLP, between 1.5 hour and 3 hours of EVLP, between 1.5 hours and 2.5 hours of EVLP or between 1 hour and 2 hours of EVLP.

19. The method of claim 1, wherein a first test EVLP perfusate sample is collected after at least or at about 45 min of EVLP, and one or more subsequent test EVLP perfusate samples are collected any time therebetween 1 min and 6 hours of collecting the first perfusate sample, optionally intervening time between collecting any two test EVLP perfusate samples is about 1, 2, 3, 4, 5, 6, 7, 8, 9, 10, 11, 12, 15, 20, 25, 30, 35, 40, 45, 50, 55, 60, 65, 70, 75, 80, 85, 90, 100, 110, 120, 150 min, optionally about 3, 3.5, 4, 4.5, 5, 5.5 or 6 hours, optionally any time therebetween 1 min and 6 hours.

20. The method of claim 1, wherein the one or more parameters values comprises at least one of a concentration of the one or more biomarkers, a rate of biomarker production of the one or more biomarkers, or a ratio of the concentration of the one or more biomarkers, and wherein the ratio is the concentration of a subsequent perfusate sample/concentration of an earlier perfusate sample, optionally wherein concentration is normalized to total lung capacity (TLC).

21. (canceled)

22. (canceled)

23. The method of claim 1, wherein the one or more biomarkers in the perfusate sample comprises or is IL-8, IL-6, sTNFR1, or sTREM-1, or wherein the one or more biomarkers in the perfusate sample comprises or are two biomarkers selected from IL-8, IL-6, sTNFR1 and sTREM-1, comprises or are three biomarkers selected from IL-8, IL-6, sTNFR1 and sTREM-1, comprises or are IL-8, IL-6, sTNFR1 and sTREM-1, are IL-8 and one or more of IL-6, sTNFR1 and sTREM-1, are IL-6 and one or more of IL-8, sTNFR1 and sTREM-1, or are IL-8 and IL-6.

24.-32. (canceled)

33. The method of claim 5, wherein the one or more parameter values is rate of IL-8 production, IL-6 production, sTNFR1 production, and/or sTREM-1 production.

34.-36. (canceled)

37. The method of claim 5, wherein the level or one or more parameter values is concentration of IL-8, IL-6, sTNFR1, and/or sTREM-1, and wherein the one or more perfusate samples is a perfusate sample taken after at least or at about 45 min of perfusion, or about 4 hours of perfusion, optionally wherein concentration normalized to total lung capacity (TLC).

38.-44. (canceled)

45. The method of claim 1, wherein the one or more parameters are at least two parameters selected from: 1) IL-6 concentration after at least or at about 1 hour of perfusion and about 4 hours of perfusion; 2) IL-8 concentration at about 4 hours of perfusion and sTNFR1 concentration at about 4 hours of perfusion; 3) IL-6 concentration after at least or at about 1 hour of perfusion and about 4 hours of perfusion and sTREM-1 after at least or at about 1 hour of perfusion; 4) IL-6 concentration after at least or at about 1 hour of perfusion and sTREM-1 concentration after at least or at about 1 hour of perfusion and IL-8 concentration at about 4 hours of perfusion, 5) sTNFR1 concentration after at least or at about 1 hour of perfusion and about 4 hours of perfusion and IL-8 concentration at about 4 hours of perfusion, 6) sTREM-1 concentration after at least or at about 1 hour of perfusion and IL-8 concentration at about 4 hours of perfusion and sTNFR1 concentration at about 4 hours of perfusion, and 7) IL-6 concentration at about 1 hour of perfusion and about 4 hours of perfusion and sTREM-1 after at least or at about 1 hour of perfusion and sTNFR1 at about 4 hours of perfusion, optionally wherein concentration is normalized to total lung capacity (TLC).

46. The method of claim 14, comprising a combination of one or more parameters, wherein the one or more parameters are at least two parameters selected from the combinations listed in FIG. 12.

47. The method of claim 1, wherein the donor lung is from a high risk donor after brain death (DBD) or a donor after cardiac death (DCD).

48. (canceled)

49. The method of claim 1, wherein the polypeptide biomarker level of the one or more biomarkers is detected using ELISA or, where more than one biomarker level is being detected, a multiplex assay.

50. A kit comprising at least one detection antibody specific for a biomarker selected from IL-8, IL-6, sTNFR1 and sTREM-1, optionally wherein the detection antibody is coupled to beads or labelled, the kit optionally further comprising one or more of a 96-well plate, standards, assay buffer, wash buffer, sample diluent, standard diluent, detection antibody diluent, streptavidin-PE, a filter plate and sealing tape, optionally for performing the method of claim 1.
